(19) United States
(12) Patent Application Publication
(10) Pub. No.: US 2019/0231639 A1
(43) Pub. Date: Aug. 1, 2019

(54) SHOCKWAVE GENERATING DEVICE AND SYSTEM

(71) Applicant: HI IMPACTS LTD, Petach Tikva (IL)

(72) Inventor: Eduard PAPIROV, Hod Hasharon (IL)

(21) Appl. No.: 16/330,110

(22) PCT Filed: Sep. 5, 2017
(86) PCT No.: PCT/IB2017/055347

§ 371 Date: Mar. 4, 2019

Related U.S. Application Data
(60) Provisional application No. 62/383,539, filed on Sep. 5, 2016.

(51) Int. Cl. A61H 23/00; F03G 1/02; A61B 17/225

(52) U.S. Cl. CPC A61H 23/008; A61B 2017/00084; A61B 17/225; F03G 1/02

(57) ABSTRACT

A shockwave generating device and system and in particular to such a device and system in which the mechanical energy stored in a spring is utilized to generate a shockwave.

### FIELD OF THE INVENTION

The present invention relates to a shockwave generating device and system and in particular to such a device and system in which the mechanical energy stored in a spring is utilized to generate a shockwave.

### BACKGROUND OF THE INVENTION

Shockwave therapy (SWT) is a non-invasive form of treatment for various medical conditions using acoustic Shockwaves. The use of shockwaves is perhaps best known for its use in fragmentation of kidney stones in a process called lithotripsy. However shockwaves have also been used for other indications such as healing bone fractures, chronic orthopedic inflammation, wound healing of chronic wounds, treatment of heart muscle ischemia as well as other medical condition as is known in the art.

Acoustic Shockwaves may be generated by a variety of force generators, including electrohydraulic electromagnetic, piezoelectric and ballistic force generators.

In ballistic shockwave generators, shockwaves are generated by high-energy collisions between two masses, with the energy propagating through a metallic media and shaped as a focused of diffused wave front starting from the geometric edge and propagating toward the treated biological tissue.

Shockwaves generating devices and system are generally associated and/or coupled with the tissue being targeted and/or treated with a fluid medium such as a gel or a water filled balloon so as to allow for the generated shockwaves to propagate and/or enter the target tissue. Therein the fluid medium is used to propagate the shockwave into the target tissue.

Shockwaves are distinct from mechanical pressure waves having specific characteristics. A pressure wave is a general term for a pressure disturbance moving through a medium. This happens to be exactly what a sound wave is. These disturbances move at the speed of sound in the medium in which they are traveling. There is no formal distinction between the two, as any amplitude of pressure wave could be heard as sound provided the listening device is sensitive enough.

A shockwave however has a specific type of pressure disturbance moving through a fluid medium. For small amplitudes, sound pressure waves pass through the medium, which then more or less returns to its initial state. However, a wave with large enough amplitude will drag a little bit of the medium along with it. That means that sound waves propagating behind it will tend to catch up with the original wave and drag the fluid behind them still faster. That process stacks up and eventually you can have a number of pressure waves that coalesce into a shockwave.

Although sharing several common properties, shockwaves differ from mechanical pressure waves in the important feature of pulse duration. The energy wavefront of true shockwaves is concentrated within several microseconds (0.25 to 4 microseconds, when measured according to IEC61846 and commonly between 0.5-1 microsecond), while the energy of a pressure wave is dispersed over several milliseconds (1 to 7 milliseconds, regularly). A shockwave pulse has a rise-time of 300 nanoseconds occurs within 1 microsecond from pulse start and a mechanical pressure pulse starts approximately 1 millisecond later.

This distinction between mechanical pressure waves and shockwaves determines the penetration of the wave energy; while mechanical pressure waves mainly affect the surface tissue, the short duration of the pressure pulse of shockwaves has limited interaction with surface tissue and the shockwaves energy propagates into the tissue and has more effect on inner body structures.

### SUMMARY OF THE INVENTION

There is an unmet need for, and it would be highly useful to have, a device and system for generating a shockwave by exploiting the potential energy stored in a spring under tension.

In embodiments, the device and system of the present invention utilize a spring under tension to release the potential kinetic energy stored therein to generate a shockwave once the tension is released. Preferably the spring is attached to a load at a first end of the spring, once the tension is released the load is accelerated against a shockwave generating surface to generate the shockwave.

Accordingly, the spring's potential energy is converted to kinetic energy of a load so as to mobilize the load against a shockwave generating surface to generate a shockwave. Thereafter the shockwave is preferably propagated from the generating surface with a shockwave propagating member comprising a fluid media for example including but not limited water, saline, gel, a fluid filled sac or the like allowing the shockwave to further propagate and penetrate further in an aqueous or fluid environment for example a body of water and/or biological tissue.

In embodiments the spring utilized are preferably a torsion spring and/or a spiral spring.

In embodiments one end of the spring is attached to a load.

In embodiments, a portion of the spring is associated with an actuator for controlling and/or determining the tension in the spring. Optionally the spring may be directly associated with actuator. Optionally the spring may be indirectly associated with an actuator for example via a coupling adaptor and/or member.

In embodiments the actuator may be provided in the form of a electric motor, DC motor, AC motor, servomotor, gear motor, or the like.

In embodiments the actuator may be provided in the form of a rotating actuator or a linear actuator.

In embodiments the device may include an electronics module comprising necessary electronics circuitry to render the device operations. In embodiments the electronic module may include at least one or more sub-modules for example including but not limited to controller sub-module, communication sub-module, power sub-module, sensor sub-module, wireless communication sub-module, wired communication sub-module, display sub-module, user interface sub-module, the like or any combination thereof.

In embodiments the power sub-module may comprise a battery, rechargeable battery, photovoltaic cells, mains power line, capacitors, super-capacitors, induction power module, the like or any combination thereof.

In embodiments, the generated shockwave generated with the device of the present invention may be utilized for any application for example including but not limited to personal use, medical use, engineering application, the like or any combination thereof.

The device according to the present invention may be configured for and/or provide a home device that is configured for home use by a user. Prior art shockwave generating devices are expensive, large and cumbersome device and system that are not conducive for user independent home use.

The present invention provides an unmet need for an extracorporeal shockwave generating device that may be safely used in the user's home setting and/or environment.

Such a device may be configured to be a compact and/or hand held device that may be independently used in a non-clinical and/or home setting by a user. For example, such home use may be utilized for various indications for example including but not limited to pain management, pain relief, wound healing, or the like or any warranted indication.

The device according to the present invention may be configured for home use for self-use such that a user may apply personal and/or self-implemented treatment. Similarly, the device may be utilized to treat an animal for example a pet, dog, cat, livestock, horse, cow, goat or the like.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is schematic block diagram of an exemplary device and system according to embodiments of the present invention.

FIG. 2A-D are schematic illustrative diagrams showing the use of an exemplary device according to embodiments of the present invention.

FIG. 3A-D are schematic illustrative diagrams showing the use of an exemplary device according to embodiments of the present invention.

FIG. 4 is schematic illustrative diagram of a device according to embodiments of the present invention.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
10 shockwaves;
50 imagery system/module;
100 shockwave device;
101 shockwave system;
102 spring;
102a first end;
102b second end;
102c connector/adaptor;
103 housing;
104 accelerated load;
105 electronic module;
106 actuator;
106a auxiliary actuator;
110 shockwave generating surface;
112 shockwave propagating member;

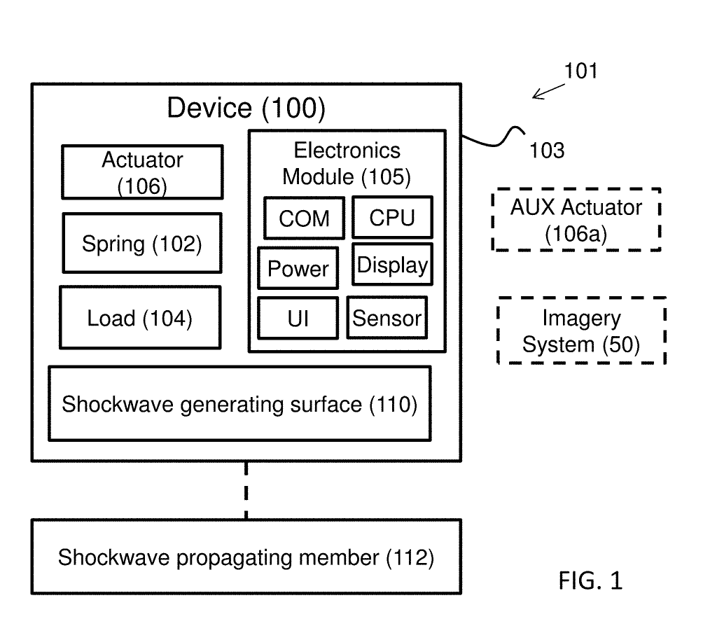

FIG. 1 shows a schematic block diagram of a device 100 that may be utilized to form a system 101 providing a spring based shockwave generating device and system.

Device 100 includes a housing 103, a spring 102, a load 104, an actuator 106, an electronics module 105, a shockwave generating surface 110. In embodiments Device 100 may further comprise a shockwave propagating member 112 that is coupled to and/or associated with surface 110.

Device 100 may form system 101 by way of further associated with auxiliary devices for example including but not limited to an imagery system 50 and an external auxiliary driving actuator 106a, any combination thereof or the like.

Device 100 provides for generating a shockwave by mobilizing a load 104, preferably a hard metallic solid and/or metal alloy, against shockwave generating surface 110, also provided from a hard metallic solid and/or alloy. The energy transferred between load 104 and surface 110 generates a shockwave that may be propagated further in an aqueous environment by propagating member 112.

In embodiments device 100 may be fit with at least one or more spring 102 and loads 104. In embodiments device 100 may be fit with at least two springs 102 each affixed with an individual load 104, that are controlled with at least one actuator 106.

In embodiments device 100 may be fit with at least two springs 102 each affixed with an individual load 104 that are controlled with a single actuator 106.

In embodiment load 104 and surface 110 may be provided from a hard metal for example including but not limited to steel, stainless steel.

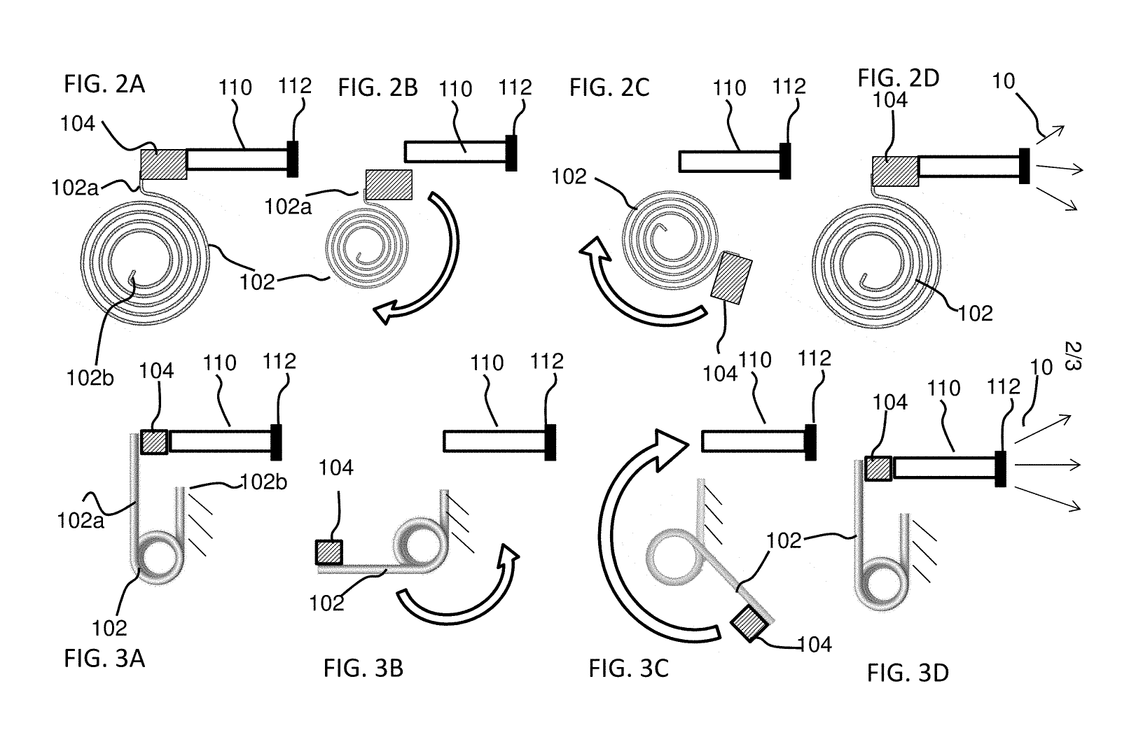

Device 100 utilizes spring 102 to drive load 104 toward surface 110. Load 104 is securely affixed to and/or coupled with an end 102a, FIG. 2-3, of spring 102. Most preferably spring 102 is provided in the form of a spiral spring, for example as shown in FIG. 2A-D, or a torsion spring, for example as shown in FIG. 3A-D. Therein the potential energy of spring 102 is converted to kinetic energy of load 104.

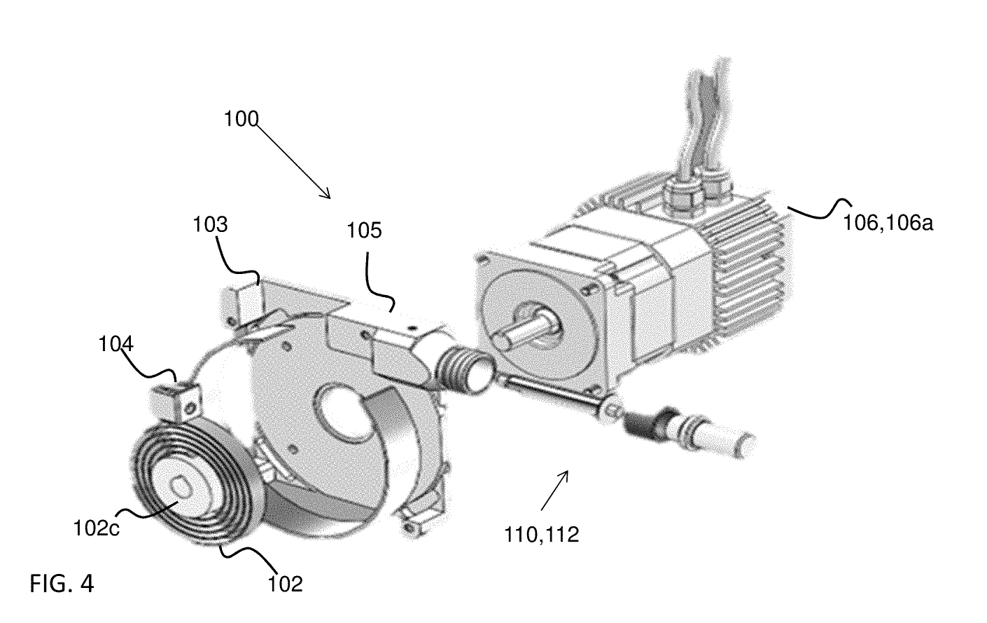

Spring 102 assumes its compressed state with the aid of actuator 106. At least a portion of spring 102 is directly or indirectly, via an adaptor, coupled with actuator 106. The compressed state of spring 102 allows device 100 to harness the spring's potential energy in converting it to the kinetic energy of load 104 associated with spring 102. Preferably actuator 106 provides for compressing and/or driving spring 102 to realize its potential. In embodiments spring 102 may be associated indirectly with actuator 106 via an adaptor 102c, for example as shown FIG. 4.

In embodiments, the shockwave energy provided by device 100 be determined by determining and/or controlling the compression level of spring 102. More preferably the compression level of spring 102 is provided and determined by actuator 106 which is controllable with electronics module 105. Accordingly device 100 may be utilized to select and/or determine the shockwave energy to be delivered by selecting the compression level of spring 102 as determined by the operation of actuator 106 associated therewith.

In embodiments actuator 106 is preferably provided in the form of an electric motor, gear motor or the like. Actuator 106 may be selected based on its ability to drive spring 102 so as to generate a shockwave.

In embodiments the actuator 106 may be provided in the form of a rotating actuator or a linear actuator.

In embodiments actuator 106 is provided in the form of a motor capable of producing about 300 revolutions per minute (RPM).

In embodiments the force of spring 102 may up to about 20 N.

In embodiments load 104 may be provided up to about 50 grams, more preferably from about 10-20 grams.

In embodiments the velocity of the spring and load could be in the range of up to 30 m/sec, or 5 to 15 m/sec, or 15-25 m/sec.

In embodiments the velocity of the spring and load is configurable based on the spring constant (k) of spring 102 and power (rpm) of actuator 106.

In embodiment shockwave generating surface 110 may be provided with at least one dimension, for example radius, diameter, length and/or thickness, of up to about 20 mm or more preferably in the range of 5 mm to about 15 mm.

In embodiments the shockwave propagating member 112 may be provided with at least one dimension, least one dimension, for example including but not limited to radius, diameter, length, and/or thickness, having a size of up to about 20 mm.

In embodiments device 100 includes an electronics module 105 comprising electronics circuitry necessary to render device 100 controllable and operational.

In embodiments electronic modules 105 may include at least one or more sub-modules for example including but not limited to controller sub-module (CPU), communication sub-module (COM), power sub-module (POWER), sensor sub-module (SENSOR), wireless communication sub-module, wired communication sub-module, display sub-module (display), user interface sub-module (UI), the like or any combination thereof.

In embodiments the power sub-module (Power) may for example include and/or comprise at least one or more of battery, rechargeable battery, photovoltaic cells, mains power line, capacitors, super-capacitors, induction power module, the like or any combination thereof.

In embodiments communication sub-module may provide for wireless and/or wired communication capabilities.

In embodiments sensory sub-module may comprise at least one or more sensors for example including but not limited to temperature sensor, pressure sensor, piezoelectric sensor, the like or any combination thereof.

Shockwave treatment device 100 comprises a shockwave surface 110 and a shockwave propagating member 112. Preferably propagating member 112 may be provided in the form of a treatment applicator and/or treatment head as is known in the art for example in the form of a fluid filled sac.

In embodiments generating surface 110 is provided from metals and/or metallic alloys, that are configured to endure and withstand impacts with load 104, and sufficient to produce shockwaves.

In embodiments generating surface 110 is shaped and sized so as to allow it to endure and withstand repeated impact with load 104, while allowing for produce shockwaves.

In embodiments generating surface 110 and load 104 may be configured relative to one another so as to maximize their mutual performance.

In embodiments shockwave generating surface 110 may be functionally associated with shockwave propagating member 112, so as to allow for the optimal transfer of shockwaves generated on surface 110 to propagate through member 112 and therefrom onto the targeted tissue.

In embodiments surface 110 is functionally associated and/or coupled with member 112 such that they are fluid and/or seamless with one another. Preferably member 112 is mechanically coupled and/or sealed with shockwave generating surface 110 to provide for efficient shockwave propagation and smooth transition therebetween.

In some embodiments of system 101 may optionally be utilized with and/or include an imagery module and/or system 50, for example medical imagery in the form of an ultrasound system, to facilitate locating and identifying a targeted treatment area. Optionally imagery system 50 may be provided in any form as is known in the art for example including but not limited to ultrasound, CT, MRI, Doppler Ultrasound, optical (laser) imagery system, any combination thereof or the like.

In some embodiments of system 101 may comprise an auxiliary actuator 106a in the form of an external motor that may be coupled to device 100 to render it functional.

In embodiments shockwave generating device 100 and/or system 101 are fit with appropriate mechanical components, sensors, electronics, controls and processing capabilities as is known in the art for shockwave generating devices, and in particular ballistic shockwave generating devices.

Now referring to FIGS. 2A-D and FIG. 3A-D showing how device 100 is utilized.

FIG. 2A-D show an embodiment of device 100 fit with a spring 102 provided in the form of spiral spring 102s. Spiral spring 102s is securely associated with a load 104.

Spring 102s is coupled with load 104 at a first end 102a, along an external surface and/or perimeter, while a second end 102b, along an internal surface and/or perimeter of spiral spring 102s is provided for coupling, directly or indirectly with actuator 106 (not shown).

FIG. 2A shows the initial conditions where spring 102,102s is at rest in its decompressed (and/or unwound) state. Load 104 is at rest and coupled to first end 102a, and spring 102s is at large diameter.

FIG. 2B shows the initiating of shockwave generation where with device 100 wherein actuator 106 (not shown) is used to wind and/or compress spring 102,102s by torqueing spring 102s at second end 102b, shown with the directional arrow. Compressing spring 102s with actuator 106 causes the reduction of the external diameter of spring 102s, as shown. As spring 102s is compressed it gains potential energy. Accordingly FIG. 2B shows the compressed mode of spring 102s. In embodiments spring 102s and load 104 may be kept at this, fully compressed, position until such a time as device 100 is ready to generate a shockwave 10. Optionally a stopper may be utilized to continuously maintain the compressed configuration.

FIG. 2C shows the initiating of the decompression of spring 102s where load 104 travels along the route provided by spring 102s therein converting the potential energy to kinetic energy as load 104 accelerates toward generating surface 110, as shown by the directional arrow. An optionally a stopper may be removed to allow for the decompression of spring 102s.

FIG. 2D shows the end the decompression of spring 102s where load 104 meets shockwave generating surface 110, causes the generation of shockwave 10 which is propagated through member 112.

A repetition of the steps shown in FIG. 2A-D cause repetition of shockwave generation by controlling how frequently spring 102s is compressed/decompressed with actuator 106.

In embodiments, device 100 is configured such that load 104 comes into contact with surface 110 when load 104 reaches its maximal velocity so as to effectively transfer the energy allowing the generation of a shockwave.

In embodiments electronics module 105 provides for controlling actuator 106 so as to control the timing and the extent of compression and decompression of spring 102,102s and load 104. Furthermore, the shockwave properties may be determined by selecting at least one or more parameter for example including but not limited to spring characteristics, spring radius, spring constant, load dimension, load mass, type of actuator, speed of actuator, strength of actuator, the like or any combination thereof.

In embodiments the speed (V) of load 104 may be determined by the spring and load characteristics load mass (m), spring speed (v), spring radius (r), spring constant (k) as given by V=ν2¶rm/k

Now referring to FIG. 3A-D, showing an embodiment that functions similarly to that described in FIG. 2A-D, however, utilizing a spring 102 in the form of a torsion spring 102t.

Spring 102t has a first end(arm) 102a that is securely affixed to load 104 and a second end (arm) 102b that is stationary providing an offset for first end 102a. Optionally second end 102b may be affixed to housing 103 and/or the like stationary portion of device 100. Torsions spring 102t is coupled and/or associated with actuator 106 (not shown) along first end (arm) 102a. Actuator 106 provides for rotating first end 102a by up to about 300 degrees to compress spring 102t so as to generate potential energy with spring 102t.

FIG. 3A shows the resting position prior to torsion of spring 102t.

FIG. 3B shows compression of spring 102t by manipulating first end 102a.

FIG. 3C shows decompression of spring 102t after it has been full compressed where first end 102a is allowed to rotate with load 104 to convert the potential energy in spring 102,102t to kinetic energy of load 104 as it meets shockwave generating surface 110 as shown in FIG. 3D, generating shockwaves 10.

As discussed above the conversion process is given by

Ep=½kX²=>X=¶r

Ek=½mV²/2 [Joule]
at maximal spring stretch/tension Ep=Ek

½kx²=½mV²

V=νkX²/m

Based on this, in embodiments the energy provided by device 100 to load 104 is up to about 10 Joules that are available of generating shockwaves with surface 110.

For example, device 100 using a load 104 having a mass of 20 grams travelling at 25 m/sec would generate 6.25 J of kinetic energy that is then converted to shockwave 10. For example, a load having a mass of 10 grams and speed of 5 m/sec would generate 0.125 J of kinetic energy that is then converted to shockwave 10. In embodiments the dimension and characteristics of generate surface 110 may be selected based on the available kinetic energy.

FIG. 4 shows a schematic depiction of device 100 as previously described with a spiral spring 102s that is coupled to an actuator 106, in the form of a gear motor, with an adaptor 102c. Spring 102,102s fits within housing 103 that has a segment for receiving spring 102, 102s. Preferably electronic module 105 is provided to control actuator 106 that in turn control the status of spring 102, load 104.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1-15. (canceled)

16. A device (100) for generating a shockwave by the conversion of mechanical potential energy of a spring (102), the device comprising;
a. a spring (102) provided in the form of a spiral spring (102s) or a torsion spring (102t), the spring having an end (102a,102b) for coupling with a load (104);
b. said spring is compressible with an actuator (106) wherein said actuator (106) is associated with an end (102a,102b) of said spring, therein provides said spring with mechanical potential energy; and
c. a shockwave generating surface (110) for generating said shockwave when said mechanical potential energy of said spring is converted to kinetic energy of said load (104) allowing said load to collide with said surface (110).

17. The device of claim 16 wherein said actuator is a motor of up to 300 rpm.

18. The device of claim 16 wherein said spring has about 20 N.

19. The device of claim 16 wherein load (104) has a mass of up to about 50 grams.

20. The device of claim 16 having a plurality of springs each associated with a load.

21. The device of claim 16 wherein kinetic energy available is up to 10 J.

22. The device of claim 16 further comprising an electronic s module (105).

23. The device of claim 22 comprising at least one or more sub-module electronic module may include at least one or more sub-modules for example including but not limited to controller sub-module, communication sub-module, power sub-module, sensor sub-module, wireless communication sub-module, wired communication sub-module, display sub-module, user interface sub-module, any combination thereof.

24. The device of claim 16 further comprising a shockwave propagating member.

25. The device of claim 24 wherein said member is a fluid filled sac.

26. A method for generating a shockwave by the conversion of mechanical potential energy to kinetic by releasing of spring when a load is accelerated to maximal radial velocity and collides with the shockwave generating surface.

27. The method of claim 26, wherein the spring comprises a spiral spring when a load is accelerated to maximal radial velocity and collides with the shockwave generating surface.

28. A system for generating a shockwave by the conversion of mechanical potential energy to kinetic by releasing of Torsion spring when a load is accelerated to maximal radial velocity and collides with the shockwave head.
